(19) United States
(12) Patent Application Publication
(10) Pub. No.: US 2002/0169140 A1
(43) Pub. Date: Nov. 14, 2002

(54) Combination therapy for reduction of toxicity of chemotherapeutic agents

(76) Inventor: Patrick T. Prendergast, Straffan (IE)

Correspondence Address: BURR & BROWN, PO BOX 7068, SYRACUSE, NY, 13261-7068, US

(21) Appl. No.: 10/091,855

(22) Filed: Mar. 6, 2002

(30) Foreign Application Priority Data
Mar. 6, 2001 (IE) S2001/0209

(51) Int. Cl. A61K 31/7076; A61K 31/522; A61K 31/353

(52) U.S. Cl. 514/45; 514/456; 514/679; 514/263.31

(57) ABSTRACT

There are provided compositions, pharmaceutical formulations and kits for treating neoplasms and tumours, viral infections, bacterial infections, or parasite infections. There are also provided compositions, pharmaceutical formulations and kits for suppression of immune response rejection in tissue transplantation. Also provided are methods of treating such conditions.

The present invention relates to a unique combination therapy for minimising dosage required of chemotherapeutic agents for their use in antiviral, antibacterial, antiparasitic and anticancer chemotherapy. This invention also relates to the use of the compounds of the present invention including precursor molecules, derivatives, metabolites, analogues, mimic molecules and to compositions containing the compounds of the present invention including precursor molecules, derivatives, metabolites, analogues, mimic molecules for use in a combination therapy with chemotherapeutic agents for their use in antiviral, antibacterial, antiparasitic and anticancer chemotherapy.

Cancer develops from changes in the DNA, or genetic material, of the body's cells, causing them to develop into precancerous lesions. Such lesions exhibit a strong tendency to develop into malignant tumours, or cancer. Such lesions include lesions of the breast (that can develop into breast cancer), lesions of the skin (that can develop into malignant melanoma or basal cell carcinoma), colonic adenomatous polyps (that can develop into colon cancer), and other such neoplasms.

Cancer may take years to develop. The process typically begins with some disruption to the DNA of a cell, the genetic code that directs the life of the cell. Many things, such as diet, tobacco, sun exposure or certain chemicals can cause such disruptions. Some cells will enter a precancerous phase, known as dysplasia. Some cells will also enter the state of carcinoma in situ, in which the cancer cells are restricted to a microscopic site and do not pose a great threat. Eventually, unless the body's own immune system takes care of the wayward cells either on its own or by being enhanced by specific chemicals, a tumour will develop. It may take as long as 30 years for a tumour to go through the entire process and become large enough to produce clinical symptoms.

Anyone can get cancer, including children, but it is most common in people over the age of 50. This year about 1.22 million people in the United States will be diagnosed with cancer (not including the more than 1 million annual cases of basal and squamous-cell skin cancers.) About 563,000 people will die of cancer this year. Treatment for cancer has progressed rapidly over the last 30 years. Doctors generally prescribe three main treatments for cancer: surgery, radiation therapy, chemotherapy or a combination of these. Choosing a course of medical treatment depends largely on the cancer type, stage of progression, and location.

Chemotherapy uses poison drugs that take advantage of cancer cells' rapid growth and consumption of large amounts of nutrients. Chemotherapy side effects include nausea, bone marrow suppression and temporary full or partial hair loss. One class of chemotherapeutic agents, the nucleotide analogues, are used as anticancer agents because they interfere with the synthesis of DNA and thereby preferentially kill rapidly dividing cells such as tumour cells. Some of the nucleotide analogues commonly used in chemotherapy are 6-mercaptopurine, 5-flourouracil, 5-iodo-2′-deoxyuridine and 6-thioguanidine. Each of these compounds disrupts the normal replication process by interfering with the formation of correct Watson-Crick base-pairing.

Physicians inject these drugs into the bloodstream, where they travel throughout the body, consumed by every cell. Rapidly growing cancerous cells consume much more of the poisonous drugs than do normal cells. As a result, the drugs destroy cancerous cells faster than normal cells. However, chemotherapy drugs act on all the patient's cells—the cancerous cells and the healthy cells. A physician's challenge is to administer the drugs to kill only the cancer cells, not the healthy cells. Unfortunately, most chemotherapeutic drugs have serious side effects that prohibit their long-term use, or use in otherwise healthy individuals with precancerous lesions. There side effects, which are a result of non-specific toxicity of the drugs, immunosuppression and other toxicities. For this reason there is a need to identify new drug candidates for therapy of patients with precancerous lesions that can be combined with existing chemotherapeutic drugs, whereby the dose can be reduced, diminishing the toxicity and serious side effects in humans.

Acquired Immunodeficiency Syndrome (AIDS) is one of the most significant infections to appear in the last decade. This epidemic is not confined to a single segment of the population nor is its spread blocked by natural barriers or international boundaries. Millions have died in Africa and many more individuals are infected worldwide. In the United States more than 100,000 people have died and at least 1 million more are presently infected with the virus. This pandemic shows no signs of abating.

AIDS was first diagnosed in male homosexuals who exhibited a variety of infections of fungal (Candida albicans), protozoal (Pneumocystis carinii), and viral (Herpes zoster) origin. Many of these individuals also had an increased incidence of kaposi sarcoma and lymphoma. They had a depressed T helper/T suppressor lymphocyte cell ratio and an absence of delayed hypersensitivity responses. Collectively, these observations suggested a deficiency in cell-mediated immunity.

It is strongly suspected that the causative agent in AIDS is an RNA retrovirus called the human immunodeficiency virus (HIV-1 or HIV-2). HIV possesses an envelope glycoprotein (gp120) that has a high affinity for the CD₄ receptor on T helper cells and other target cells. These other target cells include bone marrow stem cells, macrophages, endothelial cells, glial cells, lymph node, dendritic cells, bowel enterochromaffm cells, cervical epithelium and possibly Langerhans cells. However, it is the effects of HIV on T-helper cells that are the best known. The infectious process begins when the virus penetrates the body and enters the blood stream. Binding of HIV to CD₄ target cells involves interaction of the external envelope glycoprotein molecule gp120 with the CD₄ molecule, although other cell receptors may be involved. The virus next enters the target cell, or is internalized, through fusion of the viral envelope with the target cell membrane. Through this fusion, the virus loses its coat, and releases its RNA core and reverse transcriptase enzyme into the host cell cytoplasm.

The HIV reverse transcriptase enzyme copies the RNA message producing first a single-stranded, and then a double-stranded, DNA (circular complementary DNA). This newly formed double-stranded DNA becomes incorporated into the host chromosomal DNA once it enters the host cell nucleus. This incorporated viral DNA may remain dormant or, upon activation, will produce viral messenger RNA (mRNA). The viral mRNA codes for proteins that are important in viral replication.

Glycoprotein will then envelop the RNA genome resulting in the production of infectious viral particles; completed viral particles are then released to infect other cells.

Current approaches to HIV treatment generally involve immunotherapy (e.g., vaccines against whole killed HIV and a variety of HIV surface glycoproteins) directed at the HIV as well as pharmacological intervention in the HIV infectious process. In theory, any of the steps of viral replication or release could be points of pharmacological attack against the virus. The major chemotherapeutic attack by available drugs has been at the level of inhibition of viral reverse transcriptase using nucleotide and nucleotide analogues. The first drug licensed for use in HIV treatment became available in 1987; it was azidothymidine (AZT). In the early 1990's, dideoxyinosine (DDI) and dideoxycytidine (DDC) were approved by the FDA. AZT and DDI were approved for monotherapy while DDC is used in combination with one of the other drugs. However, when administered to patients, nucleotide and nucleoside analogues have shown toxicity to liver, bone marrow, and the nervous system. Side effects include bone marrow suppression (anemia granulocytopenia), headache, malaise, nausea, pancreatitis, peripheral neuropathy, minor GI and CNS symptoms, and oral ulcers. In the case of antiviral therapy, nucleoside analogs have been rarely curative, and the side effects that arise during chronic administration of the drug often cause therapy to be discontinued or altered. In the case of cancer therapy, where intent is to kill the cancer cells, the compound and protocol (dose, method of administration, timing of doses) must be carefully designed and monitored to minimize the damage to non-cancerous tissues.

Many different treatment regimens are and have been used to treat the HIV infection and AIDS, which occurs after the latent infection. While they might prolong survival and possibly minimize symptoms, in view of the mounting worldwide concern regarding the epidemic, these treatments have not been generally successful and cause considerable side effects. Therefore, the continuing hard reality is that once the virus enters the body and begins the uncoating process, a fatal outcome is almost inevitable. Such an outcome reveals the continuing need for additional research to discover a method of treatment, which can suppress the reproduction of latent viruses such as HIV without inducing toxicity. For this reason there is a need to identify new drug candidates for therapy of patients with HIV infection and AIDS that can be combined with existing chemotherapeutic drugs, whereby the dose can be reduced, diminishing the toxicity and serious side effects in humans.

Parasitic infections are a major worldwide health problem. The global prevalence of human parasitic infections already exceeds 50% and is increasing. Diverse factors are responsible including population crowding; poor sanitation and health education; inadequate control of parasite infections and reservoirs of infection; increased world travel; population migration and resistance to the agents used for chemotherapy or control of vectors.

Despite encouraging progress in identifying promising molecular targets for intervention with vaccines, chemotherapy remains the single most effective method to control most parasitic infections. However, to be used for mass chemotherapy, an ideal antiparasitic agent should be safe at high therapeutic doses with minimal toxicity. Few antiparasitic drugs meet these criteria. For this reason there is a need to identify new drug candidates for therapy of patients with parasitic infections that can be combined with existing chemotherapeutic drugs, whereby the dose can be reduced, diminishing the toxicity and serious side effects in humans. Furthermore, several antibiotics are not in current use doe to their toxicity profile. It would be advantageous similarly to identify new drug candidates for therapy of patients with bacterial infections that can be combined with existing chemotherapeutic drugs, whereby the dose can be reduced, diminishing the toxicity.

Additionally, the suppression of immune response to limit rejection in tissue transplantation by the use of nucleotide and nucleoside analogues is limited by the toxicity of the compounds. The identification of new drug candidates for therapy of patients with tissue transplants that can be combined with existing chemotherapeutic drugs, whereby the dose can be reduced, is necessary.

The correlation between the combination therapy including the compounds of the present invention, circiliol, precursor molecules, derivatives, metabolites, analogues and/or mimic molecules, antiviral and anticancer chemotherapy was not recognised prior to the work of the applicant. Accordingly the following provides information on each of these topics.

The present invention provides methods and pharmaceutical compositions for repressing reproduction of latent viruses, such as HIV, parasites, bacteria and for anticancer therapy in humans and animals, by the generally concurrent administration of at least one chemotherapeutic agent with compounds of the present invention, with pharmaceutical acceptable additives, diluents, carriers, excipients and pharmaceutical salts thereof.

The present invention relates to a composition comprising at least one of the compounds of the present invention.

The present invention also relates to a pharmaceutical formula comprising a composition comprising at least one of the compounds of the present invention, with pharmaceutical acceptable additives, diluents, carriers, excipients and pharmaceutical salts thereof, at least one chemotherapeutic agent and a pharmaceutically acceptable carrier. The advantage of such a pharmaceutical formula is that the antimetabolites, nucleotide and/or nucleoside analogues can be administered in much lower doses than normally administered, thereby greatly reducing their toxicity while increasing their therapeutic index. The synergistic effect of the chemotherapeutic agents in combination with the compounds of the present invention provides an enhanced efficacy despite the low dose of chemotherapeutic agent administered.

The present invention is also directed to the use of a pharmaceutical formulation for the manufacture of a medicament for treatment of a mammal suffering from a neoplasia and/or viral, bacterial and/or parasite infection and/or suppression of immune response rejection in tissue transplantation.

The present invention is also directed to a method for manufacturing a medicament for treating neoplasia and/or viral, bacterial and/or parasite infection and/or suppression of immune response rejection in tissue transplantation utilizing a specific treatment protocol.

The present invention is directed to a method of inhibiting neoplastic cells by exposing those cells to a pharmacologically effective amount of pharmaceutical formulations comprising compositions containing those compounds of the present invention with pharmaceutical acceptable additives, diluents, carriers and excipients and at least one chemotherapeutic agent. Such compounds are effective at eliminating and inhibiting the growth of neoplasias such as precancerous lesions, tumours and cancer growth. One of the advantages of utilising such compositions is that they are low in toxicity, which in combination with their mechanism of action diminishes resistance development.

The present invention provides the compounds of the present invention, circiliol, including their precursor molecules, derivatives, metabolites, analogues and/or mimic molecules with pharmaceutical acceptable additives, diluents, carriers and excipients and pharmaceutically acceptable salts thereof, as well as pharmaceutical compositions comprising the compounds of the present invention, with pharmaceutical acceptable additives, diluents, carriers and excipients and at least one chemotherapeutic agent and methods comprising inhibiting tumour growth or treating cancer by administering one or more of the compounds of the present invention, with pharmaceutical acceptable additives, diluents, carriers and excipients in combination with at least one chemotherapeutic agent.

The present invention also provides products that are useful for treating neoplasia and/or viral, bacterial and/or parasite infection and/or suppression of immune response rejection in tissue transplantation with minimal toxic side effects unlike the high toxicity associated with standard chemotherapeutic agents.

The present invention is also directed to a pharmaceutical composition comprising a biologically active amount of at least one compound of the present invention.

The present invention is also directed to providing pharmaceutical formulations for treating various neoplasias and/or viral, bacterial and/or parasite infections and/or suppression of immune response rejection in tissue transplantation.

The present invention is also directed to providing a method for treating various neoplasias and/or viral, bacterial and/or parasite infections and/or suppression of immune response rejection in tissue transplantation.

Treatment according to the present invention can be effected when the subject is a neonate. Administration is carried out prior to delivery of the neonate and/or during delivery of the neonate. Prophylactic treatment of HIV⁺ pregnant mothers prevent the viral transmission to the fetus by being able to administer lower less toxic levels of chemotherapeutic agents in combination with the compounds of the present invention.

These and other objects of the present invention will become apparent from the description of the invention disclosed below, which descriptions are intended to limit either the spirit or scope of the invention but are only offered as illustrations of the preferred embodiments of the invention.

The present invention is directed to the treatment, inhibition and/or prevention of viral, bacterial and/or parasite infections and/or suppression of immune response rejection in tissue transplantation and/or tumours and/or cancer growth and more particularly to treating neoplasia and/or HIV.

The invention features a method of treating neoplasia and/or viral, bacterial and/or parasite infection and/or suppression of immune response rejection in tissue transplantation, which includes administering to an animal an effective amount of a pharmaceutical formulation comprising a composition comprising at least one of the compounds of the present invention, with pharmaceutical acceptable additives, diluents, carriers, excipients and pharmaceutical salts thereof, at least one chemotherapeutic agent and a pharmaceutically acceptable carrier.

The invention is directed to a method of treating tumours and/or viral, bacterial and/or parasite infections and/or suppression of immune response rejection in tissue transplantation comprising administering a biologically active amount of a pharmaceutical formula comprising a composition comprising at least one of the compounds of the present invention, with pharmaceutical acceptable additives, diluents, carriers, excipients and pharmaceutical salts thereof, at least one chemotherapeutic agent and a pharmaceutically acceptable carrier.

The invention features a method of treating cancer and/or viral, bacterial and/or parasite infections and/or suppression of immune response rejection in tissue transplantation comprising administering to a patient in need thereof a cancer and/or viral, bacterial and/or parasite treatment and/or suppression of immune response rejection in tissue transplantation an effective amount of a pharmaceutical formula comprising a composition comprising at least one of the compounds of the present invention, with pharmaceutical acceptable additives, diluents, carriers, excipients and pharmaceutical salts thereof, at least one chemotherapeutic agent and a pharmaceutically acceptable carrier.

The inventions objects include the provision of pharmaceutical formulations and compounds, which are suitable for making the formulations. Other objects are to provide methods to use the formulations.

It was surprisingly found that when the compounds of the present invention were administered in combination with chemotherapeutic agents, the proliferation of neoplastic cells was inhibited, which is manifested, pursuant to one aspect of the present invention, in a broad-spectrum anti-neoplastic activity with minimal toxicity. The compounds of the present invention are individually diverse, but collectively all act to inhibit the propagation of neoplastic cells, cancers, cancer growth and/or tumours.

The invention also features a method of treating neoplasia and/or viral, bacterial and/or parasite infection and/or suppression of immune response rejection in tissue transplantation comprising administering to a patient an effective amount of a pharmaceutical composition including at least one compound of the present invention, at least one chemotherapeutic agent and a pharmaceutically acceptable carrier.

Treating neoplasia in a patient includes achieving, partially or substantially, one or more of the following: arresting the growth or spread of a cancer, reducing the extent of a cancer (e.g., reducing size of a tumour or reducing the number of affected sites), inhibiting the growth rate of a cancer, and ameliorating or improving a clinical symptom or indicator associated with a cancer (such as tissue or serum components).

In accordance with the present invention, a method is provided to treat or prevent viral, bacterial and/or parasite infection and/or suppression of immune response rejection in tissue transplantation comprising administering to a subject an effective amount of one or more of the compounds of the present invention, with pharmaceutical acceptable additives, diluents, carriers and excipients and pharmaceutically acceptable salts thereof in combination with a chemotherapeutic agent.

The present invention also provides the use of one or more of the compounds of the present invention, with pharmaceutical acceptable additives, diluents, carriers and excipients and pharmaceutically acceptable salts thereof in combination with a chemotherapeutic agent, for the manufacture of a medicament for a viral, bacterial and/or parasite infection and/or suppression of immune response rejection in tissue transplantation.

The present invention also provides compounds for use in a method of treatment of a viral, bacterial and/or parasite infection and/or suppression of immune response rejection in tissue transplantation, said method comprising administering one or more to a subject.

### DETAILED DESCRIPTION OF THE INVENTION

The compounds of the present invention comprise circiliol and corresponding pharmaceutically acceptable precursor molecules, derivatives, metabolites, analogues, mimic molecules and mixtures thereof,

The present invention relates to a composition comprising one or more of the compounds of the present invention, with pharmaceutical acceptable additives, diluents, carriers, excipients and pharmaceutical salts thereof.

In another embodiment of the invention, the composition includes but is not limited to one or more of the following components: Circiliol; 6,7-dimethoxy-5,3′4′-trihydroxyflavone; 6-hydroxy-2,3,4-trimethoxy acetophenone; 2-(3,4-dibenzyloxybenzoyloxy) 4,5,6-trimethoxy acetophenone; 3′4′-dibenyloxy-2-hydroxy-4,5,6-trimethoxydibenzoyl methane; 6,7-dibenzyloxy-5,6,7-trimethoxy flavone; 3,4-dihydroxy-5,6,7-trimethoxy flavone; 3,4-diacetoxy-5,6,7-trimethoxy flavone, their derivatives, metabolites, analogues and/or mimic molecules with pharmaceutical acceptable additives, diluents, carriers and excipients and pharmaceutically acceptable salts thereof.

In one embodiment, the chemotherapeutic agents are antimetabolites, nucleotide and/or nucleoside analogues. Novel antimetabolites, nucleotide and/or nucleoside analogues are disclosed in U.S. Pat. Nos. 6,127,540; 5,837,871; 5,879,700; 5,977,061; 5,981,507; 5,994,321; and 6,071,922 and are incorporated by reference.

In another embodiment the nucleotide analogues are pyrimidine antagonists, preferably selected from but not limited to the group comprising: 5-flourouracil; cytosine arabinoside; Azacitidine. In another embodiment the nucleotide analogues are purine antagonists, preferably selected from but not limited to the group comprising: 6-mercaptopurine; azathioprine; 5-iodo-2′-deoxyuridine; 6-thioguanine; 2-deoxycoformycin, cladribine, cytarabine, fludarabine, mercaptopurine, thioguanine, pentostatin.

In another embodiment the nucleoside analogues are preferably selected from but not limited to the group comprising: purine and pyrimidine nucleosides; ara-nucleosides; amino-nucleosides; aza-nucleosides; In another embodiment the nucleoside analogues are preferably selected from but not limited to the group comprising: AZT (zidovudine); ACV; valacylovir; famiciclovir; acyclovir; cidofovir; penciclovir; ganciclovir; Ribavirin; ddC; ddl (zalcitabine); lamuvidine; Abacavir; Adefovir; Didanosine; gemcitabine; d4T (stavudine); 3TC; BW 1592; PMEA/bis-POM PMEA; ddT, HPMPC, HPMPG, HPMPA, PMEA, PMEG, dOTC; DAPD; Ara-AC, pentostatin; dihydro-5-azacytidine; tiazofurin; sangivamycin; Ara-A (vidarabine); 6-MMPR; 5-FUDR (floxuridine); cytarabine (Ara-C; cytosine arabinoside); 5-azacytidine (azacitidine); HBG [9-(4-hydroxybutyl)guanine], (1S,4R)-4-[2-amino-6-cyclopropyl-amino)-9H-purin-9-yl]-2-cyclopentene-1-methanol succinate (“159U89”), uridine; thymidine; idoxuridine; 3-deazauridine; cyclocytidine; dihydro-5-azacytidine; triciribine, ribavirin and fludrabine. In another embodiment, the chemotherapeutic agents selected from the group but are not limited to: Chloroquin, primaquine, mefloquine, pyrimethamine-sulfadoxone, atoraquone/dapsone; halofantrine; artemisinin derivatives; atoraquone+proguanol, co-artemether; podophyllotoxin; pentamidine, diloxanide furoate, metronidazole, tindazole, tetracycline, quinacrine, stibogluconate, amphotericin B, quinine, doxycline, trimethoprim-sulfamethoxazole, metronidazole, nifurtimox, suramin, melarsoprol, benznidazole, their metabolites, salts derivatives or any other anti-parasitic agent thereof.

In another embodiment, the chemotherapeutic agents selected from the group but are not limited to: mitomycin C, nalidixic acid, puromycin, sanamycin, and actinomycin.

In another embodiment, the chemotherapeutic agents selected from the group but are not limited to: N⁶-(Δ²-isopentyl) adenosine, N⁶-(Δ²-isopentyl) adenosine-5′-monophosphate, N⁶-(Δ²-isopentyl) adenosine-3′,5′-cyclic monophosphate, benzyladenosine, N⁶-benzyladenosine-5′-monophosphate, N⁶-benzyladenosine-3′,5′-cyclic monophosphate, furfuryladenosine, N⁶-furfuryladenosine-5′-monophosphate, N⁶-furfuryladenosine-3′,5′-cyclic monophosphate, N-(purin-6ylcarbamoyl)-o-chloroaniline ribonucleoside, N-(purin-6ylcarbamoyl)-o-chloroaniline ribonucleoside-5′-monophosphate, N⁶-adamantyladenosine, N⁶-adamantyladenosine-5′-monophosphate, N-(purin-6ylcarbamoyl)-o-octylamine ribonucleoside, N-(purin-6ylcarbamoyl)-o-octylamine ribonucleoside-5′-monophosphate, N-(purin-6ylcarbamoyl)-o-octylamine ribonucleoside-3′,5′-cyclic monophosphate, N⁶-(Δ²-isopentyl)-2-methylthioadenosine, N⁶-(4-hydroxy-3methyl-trans-2-butenyl)adenosin, N⁶-(3-chloro-trans-2-butenyl) adenosine, surfinal adenosine and preferred metabolites including N⁶-(Δ²-isopentyl) adenine, 6-N-(3-methyl-3-hydroxybutylamino) purine, adenine, hypoxanthine, uric acid and methylated xanthines.

In a further or alternative aspect of the invention, there is provided a method for the treatment of a viral, bacterial and/or parasite infection and/or suppression of immune response rejection in tissue transplantation comprising the step of administering an effective dose of a compound of the present invention as defined hereinabove or a pharmaceutically acceptable derivative thereof combined with at least one chemotherapeutic agent. By combining the compounds of the present invention with chemotherapeutic agents a synergistic effect is observed and a much lower dosage of the chemotherapeutic agents is required, achieving the desired therapeutic effect while limiting the toxicity to the patient.

As will be appreciated by those skilled in the art, references herein to treatment extends to prophylaxis as well as to the treatment of established infections of symptoms.

By the term “pharmaceutically acceptable derivative” is meant any pharmaceutically acceptable salt, ester, or salt of such ester of a compound of the present invention, or any other compound which, upon administration to the recipient, is capable of providing (directly or indirectly) a compound of the present invention or an active metabolite or residue thereof.

Pharmaceutically acceptable salts of the compounds of the present invention include those derived from pharmaceutically acceptable inorganic and organic acids and bases. Examples of suitable acids include hydrochloric, hydrobromic, sulphuric, nitric, perchloric, fumaric, maleic, phosphoric, lactic, salicylic, succinic, p-toluenesulphonic, tartaric, acetic, citric, methanesulphonic, formic, benzoic, malonic, naphtalene-2-sulphonic and benzenesulphonic acids. Other acids such as oxalic, while not in themselves pharmaceutically acceptable, may be useful in the preparation of salts useful as intermediates in obtaining the compounds of the invention and their pharmaceutically acceptable acid addition salts. Salts derived from appropriate bases include alkali metal (e.g. sodium), alkaline earth metal (e.g. magnesium), ammonium and N(R′).sub.4.sup.+(where R′is C.sub.1-4alkyl) salts.

In a preferred embodiment, the nucleoside analogue is a phosphate ester selected from the group comprising: Acyclovir; 1-beta-D-arabinofuranosyl-E-5-(2-bromovinyl) uracil; 2′-fluorocarbocyclic-2′-deoxyguanosine; 6′-fluorocarbocyclic-2′-deoxyguanosin; 1-(beta-D-arabinofuranosyl)-5(E)-(2-iodovinyl)uracil; {(1r-1.alpha., 2.beta., 3.alpha.)-2-amino-9-(2,3-bis(hydroxymethyl)cyclobutyl)-6H-purin-6-one}Lobucavir; 9H-purin-2-amine, 9-((2-(1-methylethoxy)-1-((1-methylethoxy) methyl)ethoxy)methyl)-(9Cl); trifluorothymidine; 9>-(1,3-dihydroxy-2-propxy) methyl)methyllguanine (ganciclovir); 5-ethyl-2′-deoxyuridine; E-5-(2-bromovinyl)-2′-deoxyuridine; 5-(2-chloroethyl)-2′-deoxyuridine; buciclovir; 6-deoxyacyclovir; 9-(4-hydroxy-3-hydroxymethylbut-1-yl)guanine; E-5-(2-iodovinyl)-2′deoxyuridine; 5-vinyl-1beta-D-arabinofuranosyluracil; 1-beta-D-arabinofuranosylthymine; 2′-nor-2′deoxyguanosine; 1-beta-D-arabinofuranosyladenine.

In another embodiment, the nucleoside phosphate ester is in the form of a pharmaceutically acceptable salt. In another embodiment, the salt is selected from the group consisting of sodium, potassium, and hydrogen salts.

in certain embodiments the compounds of the present invention and chemotherapeutic agents are combined with a physiological carrier for the treatment of the pathophysiological state.

In another embodiment the pharmaceutical formulation is delivered to infected cells by incorporating the pharmaceutical formulation into liposomes or carbohydrate vehicles.

In another embodiment the pharmaceutical formulation further includes a pharmaceutically acceptable carrier, which in one embodiment is a cyclodextrin, alpha-cyclodextrin, beta-cyclodextrin, (beta-hydroxypropylcyclodextrin) gamma-cyclodextrin and in another embodiment is vitamin E oil, DMSO and/or ethanol. The compounds of the present invention can be formulated and administered as free bases or in the form of their pharmaceutically acceptable salts for purposes of stability, convenience of crystallisation, increased solubility, and the like.

As used herein, the term “neoplasia” or neoplasm covers dysplasia, precancerous lesions, cancerous lesions, neoplastic cells, cancer, cancer growth, tumours, benign tumours, malignant tumours, solid tumours, carcinomas, etc.

As used herein, the term “precancerous lesion” includes syndromes represented by abnormal neoplastic, including dysplastic, changes of tissue. Examples include precancerous growths in colonic, breast, renal, central nervous, gastric, or lung tissues, or conditions such as dysplastic nevus syndrome, a precursor to malignant melanoma of the skin. Examples also include, in addition to dysplastic nevus syndromes, polyposis syndromes, colonic polyps, precancerous lesions of the cervix (i.e., cervical dysplasia), prostatic dysplasia, bronchial dysplasia, breast, bladder and/or skin and related conditions (e.g., actinic keratosis), whether the lesions are clinically identifiable or not.

The pharmaceutical formulations of the present invention can be administered to a mammal having a susceptible cancer, i.e. a malignant cell population or tumour. The combination of the compounds of the present invention and chemotherapeutic agents are effective on human tumours in vivo as well as on tumour cell lines in vitro. The pharmaceutical formulations of the present invention may be particularly useful for the treatment of solid tumours for which relatively few treatments are available. Such tumours include epidermoid and myeloid tumours, acute or chronic, nonsmall cell, squamous. Specific cancers which may be mentioned as susceptible to treatment by administration of pharmaceutical formulations in accordance with the present invention include prostate cancer, colon cancer, small cell lung cancer, large cell lung cancer, lung adenocarcinoma, epidermoid lung cancer, melanoma (including amelanotic subtypes), renal cell carcinoma, gastric carcinoma, cancers of the central nervous system (based on the likelihood that the compounds will cross the blood cell barrier) including brain tumours, neuroblastomas, gastric carcinoma, breast cancer, ovarian cancer, testicular cancer, lymphoma and leukaemia, oesophageal cancer, stomach cancer, liver cancers, prostate cancer, cervical cancer, adrenal cancer, oral or mucosal cancer, bladder cancer, pancreatic cancer, lymphoma, Hodgkins disease, sarcomas. Haematopoietic cell cancers such as B cell leukaemia/lymphomas, myelomas, T-cell leukemias/lymphomas, small cell leukemias/lymphomas, null cell, sezary, monocytic, myelomonocytic and Hairy cell leukemias. These lymphomasileukemias can be either acute or chronic. Other cancers may also be susceptible to treatment with the compounds of the present invention. The activity can readily be measured using standardised tests known to those skilled in the art.

As used herein, the term “carcinomas” refers to lesions that are cancerous. Examples include malignant melanomas, breast cancer, and colon cancer. As used herein, the term “neoplasm” refers to both precancerous and cancerous lesions.

As used herein, the terms “inhibit” or “inhibiting,” mean decreasing tumour cell growth rate from the rate that would occur without treatment and/or causing tumour mass to decrease. Inhibiting also includes causing a complete regression of the tumour. Thus the compounds of the present invention can be either cytostatic or cytotoxic to the tumour cells.

In another embodiment, the subjects viral infection is selected from a DNA virus infection or an RNA virus. In a preferred embodiment, the DNA virus infection or the RNA virus infection is selected from a HIV, SHIV, SIV, FIV, HSV, CMV, HAV, HBV, HCV, HDV, HEV, EBV, BVDV, HSV-1, HSV-2, HSV-6, HHV-6, HHV-8, retrovirus infection, a togavirus infection, a flavivirus infection, a rubivirus infection, a pestivirus infection, a lipid envelope virus infection, a filovirus, a picornavirus infection, a rhinovirus infection, a coronavirus infection, a respiratory syncytial virus infection, a poliovirus infection, a parainfluenza virus infection, influenza virus infection, hantavirus, adeno-associated virus, measles virus, poxvirus, filovirus, human papilloma virus and animal papilloma virus infection.

In another embodiment, the patient is suffering from one or more complications or co-infections associated with AIDS, AIDS related syndromes, including cachexia and/or wasting syndrome.

In another embodiment, the parasite infection is a Trypanosoma, Plasmodium, Cryptosporidium, Entamoeba, Balantidium, Leishmania, Pneumocystis, Trichomoniasis or Toxoplasma parasite infection.

The present invention also relates to the administration of a pharmaceutical formulation to a patient infected with a retrovirus or is suffering with AIDS and co-infected with Trypanosoma, Plasmodium, Cryptosporidium, Entamoeba, Balantidium, Leishmania, Pneumocystis, Trichomoniasis or Toxoplasma parasites or a Mycoplasma bacterium.

Another aspect of the present invention is the effective treatment of malaria, sleeping sickness, African trypanosomiasis, Chagas disease, American trypanosomiasis, cryptosporidiosis, amebiasis, balantidiasis, giardiasis, leishmaniasis, pneumocystosis, trichomoniasis, and toxoplasmosis with the pharmaceutical formulation of the present invention.

Accordingly, the present invention contemplates administering daily to a subject an amount of pharmaceutical formulation of the present invention that is clinically effective in treating or preventing a parasite infection, which the subject suffers or is at risk from infection. Illustrative parasites against which the invention can be applied are Trypanosoma, Plasmodium, Cryptosporidium, Entamoeba, Balantidium, Leishmania, Pneumocystis, Trichomoniasis or Toxoplasma parasites wherein the Trypanosoma parasites are selected from the group, but is not limited to Trypanosoma cruzi, Trypanosoma brucei, Trypanosoma gambiense, Trypanosoma rhodesiense, wherein the Plasmodium parasites are selected from the group, but is not limited to Plasmodium falciparum, Plasmodium vivax, Plasmodium malariae, Plasmodium ovate, Plasmodium berghei, wherein the Entamoeba is Entamoeba histolytica, wherein the Balantidium is Balantidium coli, wherein the Leishmania is selected for the group, but is not limited to Leishmania brazilienis, Leishmania mexicana, Leishmania donovani, Leishmania tropica, wherein the Pneumocystis is Pneumocystis carinii, wherein the Trichomoniasis is Trichomoniasis vaginalis, and the Toxoplasma parasite is Toxoplasma gondii, among other parasites.

In another embodiment, the bacterial infection is an intracellular bacterial infection, an extracellular bacterial infection, a mycoplasma infection, a Listeria infection or a Mycobacterium infection; a Streptococcus infection, a Staphylococcus infection, a Vibrio infection, a Salmonella infection; a Shigella infection, an enterotoxigenic, enteropathogenic, enteroinvasive or enterohemorrhagic E. coli infection, a Yersinia infection, a Campylobacter infection, a Pseudomonas infection, a Borrelia infection, a Legionella infection and a Haemophilus infection; pulmonary Aspergillosis, mucosal or oropharyngealcandidiasis and juvenile paracoccidiomyosis; or any combination of the above.

In another embodiment, the composition further includes a pharmaceutically acceptable carrier.

In another embodiment, a pharmaceutical formulation or method in the prophylaxis and therapy of viral infections or a complication or consequence thereof is provided. In particular the invention relates to the use a pharmaceutical formulation of the present invention, in the prophylaxis and therapy of viral infections, viral replication and the development and prevention of the deficiency of the immune system resulting in the development of opportunistic infections and certain cancers. More especially the invention relates to the use of a pharmaceutical formulation of the present invention, in the prophylaxis and therapy of viral infections, an example of which is the retrovirus, thought to be responsible for the Acquired Immune Deficiency Syndrome (AIDS) and AIDS related syndromes, believed to result from infection from the Human Immunodeficiency Virus (HIV), antibodies to which are found in almost all individuals diagnosed with AIDS.

Other embodiments provide a pharmaceutical formulation or method to treat a viral infection or to ameliorate one or more symptoms associated with a viral infection such as a flaviviral or retroviral infection comprising administering to an infected patient an effective amount of a pharmaceutical formulation as disclosed herein. It is an object of the present invention to provide a pharmaceutical formulation of the present invention for treatment, therapeutic or prophylaxis, against a viral, bacterial and/or parasitic infection. The compounds of the present invention are synergistic agents that enhance the efficacy of the chemotherapeutic agents, enabling their administration at much lower doses to achieve the therapeutic benefit, while minimizing the toxicity.

Accordingly disclosed herein is a pharmaceutical formulation or a method for treating a viral, parasite and/or bacterial infection and/or suppression of immune response rejection in tissue transplantation; for use in treating any infection, preventing a future infection and/or minimizing the effects of a future infection by a virus, bacteria and/or parasite comprising administering to a patient in need thereof a prophylactically or therapeutically effective amount of a composition comprising at least one of the compounds of the present invention, with pharmaceutical acceptable additives, diluents, carriers, excipients and pharmaceutical salts thereof, at least one chemotherapeutic agent and a pharmaceutically acceptable carrier.

The advantage of this is that an effective anti-viral is provided that has minimal risk of conferring resistance and incurring toxicity.

In one embodiment of the invention the infection is a viral infection, in another embodiment the viral infection is caused by a retrovirus, in another embodiment, the retroviral infection is caused by HIV or AIDS virus, Herpes virus, cytomegalovirus, or an animal virus, and in an additional embodiment the viral infection is caused by a lipid envelope virus.

In another embodiment, the pharmaceutical formulation is used to treat AIDS related syndromes, including cachexia and/or wasting syndrome.

One useful property of the pharmaceutical formulation of the present invention is their anti-viral activity, which is manifested, pursuant to one aspect of the present invention, in a broad-spectrum anti-viral activity. Accordingly, the present invention contemplates administering daily to a subject an amount of a pharmaceutical formulation of the present invention that is clinically effective in treating or preventing a viral infection, which the subject suffers or is at risk from infection. Illustrative viruses against which the invention can be applied are HIV, cytomegalovirus (CMV), a KS-producing herpes virus, Kaposi's Sarcoma-associated herpes virus, the virus of the genus hepatitis, a virus of the genus picornaviruses, a virus of the genus molluscipoxvirus, hantaviruses, among other viruses.

Additionally, the present invention is also broadly directed providing the use of the pharmaceutical formulation of the present invention in the treatment (i.e., in the sense of treating an existing infection, preventing a future infection, minimizing the effect of a future infection and/or enhancing the performance of a vaccine) of all infections which are not retroviral infection, several representative examples of which include one or more kind of Mycoplasma, and/or one or more diseases caused by Mycoplasmas and/or one or more of the following infections: hairy leukoplakia, oral candidosis, mouth ulcerations—aphthous/herpatic/bacterial, fungal candida, squamous oral carcinoma, Kaposi's sarcoma oral lesions, periodontitis, necrotizing gingivitis, human papilloma virus, rhinovirus and arboviral molluscum contagiosum, orafacial herpes zoster, Epstein barr virus, rotaviruses, togaviruses, including alpha viruses (also known as arboviruses, group A), flaviviruses (also known as arboviruses, group B, such as yellow fever, as well as hepatitis C and hepatitis G), rubiviruses (also known as rubella viruses e.g., human rubella virus), pestiviruses (also known as mucosal disease viruses, such as bovine virus diarrhorea virus BVDV, hog cholera virus, and sheep border disease), as well as any other non-retroviral viral induced infections. Thus virus infections that may be treated include but are not limited to HIV, SIV, FIV, FELV, SHIV, Kaposi's Sarcom-associated herpes virus and other herpes viruses (e.g. HSV-1, HSV-2, human herpes virus 6 (HHV-6) and HHV-8), the viruses associated with hepatitis (HAV, HBV, hepatitis C virus [HCV]), and human cytomegalovirus, togaviruses and flaviviruses, e.g., California encephalitis virus, St. Louis encephalitis virus, western equine encephalitis virus, eastern equine encephalitis virus, Colorado tick fever virus, LaCrosse encephalitis virus, Japanese encephalitis virus, yellow fever virus, Venezuelan equine encephalitis virus, Murray valley fever virus, tick-borne encephalitis virus, GB virus A, GB virus B, GB virus C, Dengue virus 1, Dengue virus 2, Dengue virus 3, Dengue virus 4, Semliki Forest virus, Sinbis virus, picornaviruses, rhinoviruses, coronaviruses, respiratory syncytial viruses, polioviruses, parainfluenza viruses and influenza viruses ( including type A, type B, and type C.

Pursuant to a preferred embodiment of the invention, an effective amount of a pharmaceutical formulation of the present invention thus administered is such as to produce a circulating concentration of the compounds of the present invention, including precursor molecules, derivatives, metabolites, analogues, mimic molecules, and at least one chemotherapeutic agent sufficient to reduce viral loads as monitored by, e.g., viral titre methods or by PCR.

According to a further aspect of the invention the pharmaceutical formulations is formulated in a liposome.

In another embodiment of the invention, liposomes are provided carrying the pharmaceutical formulations of the present invention targeted to HIV infected cells by putting antibodies to the HIV coat protein gp160 or gp120 on its surface. The advantage of this is that the liposome can selectively target HIV infected cells.

As used herein, the terms subject and patient are used interchangeably. Subjects and patients are mammals.

The pharmaceutical formulations of the present invention are useful antineoplastic agents i.e. to inhibit tumour cell growth in vitro and in vivo, in mammalian hosts, such as humans or domestic animals, and are particularly effective against solid tumours and multidrug resistant tumours. Thus the invention provides a method comprising inhibiting cancer cells, by contacting said cells, in vitro and in vivo with an effective amount of a pharmaceutical formulation of the present invention. The invention also provides a therapeutic method comprising treating cancer (i.e. inhibiting tumour cell growth) by administering a pharmaceutical formulation of the present invention to a mammal in need of such therapy.

The present invention is also directed to pro-drug compounds analogous to the active compounds disclosed herein. Such compounds are generally themselves inactive or low in activity, but are converted into active compounds. Thus, for example, pro-drugs such as the methyl ester of any acid functionality, which is not active per se or has very low activity could be hydrolysed, either uncatalytically or catalytically with an enzyme such as an esterase, to an active compound. Such pro-drug compounds could well be the preferred therapeutic form of the present compounds. These analogous prodrugs can be produced from active compounds based on procedures and factors that are well known to one of ordinary skill in the art. Accordingly as used in the present application, “pro-drug analogue” means “a chemical which is relatively non-toxic and pharmacologically inert but which can be transformed in vivo to a pharmacologically active drug”. More specifically it means a precursor molecule, derivative, metabolite or analogue of the compounds of the present invention which have low or no ability as antineoplastic, antiviral, antibacterial, antiparasitic agents until converted in the body to a derivative, metabolite or analogue with such ability or abilities. Such pro-drugs should have favourable properties such as enhanced absorption, water solubility, lower toxicity, or better targeting to the tumour cell (such as by reason of greater affinity to the tumour cell or a larger quantity of activating enzyme in the tumour cell as opposed to a normal cell so that larger concentrations of the active compound are produced in the tumour cell). Examples of such compounds are esters, such as methyl, ethyl, phenyl, N,N-dimethylaminoethyl, acyl derivatives such as benzoyl, p-N,N-dimethylaminobenzoyl, N,N-dimethylaminoglycyl, peptide derivatives such as γ-glutamyl, glycyl, D-Val-Leu-Lys.

The pharmaceutical formulation according to the present invention can be administered to a patient in any of a wide range of routes. Thus, with regard to the types of formulations in which the active compounds according to the present invention can be administered, as well as any additives can be included with the active compounds in the formulations, and the possible routes of administration, it is well known to those of skill in the art that such formulations can be provided in a wide variety of types, and it is within the skill of the ordinary artisans to select a specific formulation and route of administration and then test suitability for use. By way of example but not limitation, suitable routes include enteric, parenteral, topical, oral, rectal, nasal or vaginal routes. Parenteral routes include subcutaneous, intramuscular, intravenous, intraperitoneal, intradermal and sublingual administration. Also, compositions may be implanted into a patient or injected using a drug delivery system.

The pharmaceutical formulation according to the present invention may be administered locally or systemically. By systemic administration means any mode or route of administration that results in effective amounts of active ingredient appearing in the blood or at a site remote from the route of administration of the active ingredient.

Further, the pharmaceutical formulation according to the present invention may be administered intermittently. The advantage of this is that it allows the patient to suspend therapy for periods without the worry of inactivity of the drug resulting from the development of resistant cells.

The pharmaceutical formulation according to the invention may be formulated for enteral, parenteral or topical administration. Indeed all three types of formulations may be used simultaneously to achieve systemic administration of the active ingredient.

Compounds useful in the methods of this invention may be formulated into compositions together with pharmaceutically acceptable carriers for oral administration in solid or liquid form, or for rectal administration, although carriers for oral administration are most preferred.

Pharmaceutically acceptable carriers for rectal administration are preferably suppositories that may contain, in addition to the compounds of the present invention, excipients such as cocoa butter or a suppository wax.

Pharmaceutically acceptable carriers for oral administration include capsules, tablets, pills, powders, troches and granules. In such solid dosage forms, the carrier can comprise at least one inert diluent such as sucrose, lactose or starch. Such carriers can also comprise, as is normal practice, additional substances other than diluents, e.g., lubricating agents such as magnesium stearate. In the case of capsules, tablets, troches and pills, the carriers may also comprise buffering agents. Carriers such as tablets, pills and granules can be prepared with enteric coatings on the surfaces of the tablets, pills or granules. Alternatively, the enterically coated compound can be pressed into a tablet, pill, or granule, and the tablet, pill or granules for administration to the patient. Preferred enteric coatings include those that dissolve or disintegrate at colonic pH such as shellac or Eudraget S. Additional pharmaceutically acceptable carriers include liquid dosage forms for oral administration, e.g. pharmaceutically acceptable emulsions, solutions, suspensions, syrups and elixirs containing inert diluents commonly used in the art, such as water. Besides such inert diluents, compositions can also include adjuvants such as wetting agents, emulsifying and suspending agents, and sweetening, flavouring and perfuming agents.

In one embodiment, the pharmaceutical formulation has an enteric coating. In another embodiment, the enteric coating is made of a polymer or copolymer. In a preferred embodiment, the polymer or copolymer is selected from the group consisting of poly(lactic-glycolic acid) polyester, cellulose acetate phthalate, hydroxypropyl-methyl cellulose phthalate poly(butyl methacrylate), (2-dimethyl aminoethyl) methacrylate, and methyl methacrylate.

In accordance with the present invention, the expression “micronized” means that the compound has been micronized in accordance with any process for micronizing, a number of which are known in the art. The micronized particles preferably include a percentage of particles, which are of a diameter, which is about 10 microns, or less, preferably, 5 microns or less. For example, in a preferred aspect of the invention, at least 80% of the particles in a formulation of micronized particles have a diameter of less than 5 microns. An alternative to micronizing a compound is to solubilize the compound and put it into liposomes of appropriate size. The manufacture of liposomes and the insertion of active ingredients into such liposomes are well known in the art.

Suitable injectable solutions include intravenous, subcutaneous and intramuscular injectable solutions. Examples of injectable forms include solutions, suspensions and emulsions. Typically the compound(s) is injected in association with a pharmaceutical carrier such as normal saline, Ringers solution, dextrose solution and other aqueous carriers known in the art. Appropriate non-aqueous carriers may also be used and examples include cyclodextrin, preferably hydroxypropyl beta cyclodextrin, mixed oils (vitamin E oil), polyethylene glycol and ethyl oleate. A preferred carrier is cyclodextrin in water. Frequently, it is desirable to include additives in the carrier such as buffers and preservatives or other substances to enhance isotonicity and chemical stability.

The pharmaceutical formulation can also be administered topically. Suitable formulations for topical administration include creams, gels, jellies, mucliages, pastes and ointments. The compounds may be formulated for transdermal administration, for example in the form of transdermal patches so as to achieve systemic administration.

The pharmaceutical formulation may also be administered in the form of an implant.

The pharmaceutical formulation may also be administered in the form of an infusion solution or as a nasal inhalation, aerosol or spray.

In another embodiment, the composition is incorporated in a pharmaceutically acceptable carrier, diluents, vehicles and the like for systemic administration by feeding. An example of such a carrier is cyclodextrin (α-cyclodextrin, β-hydroxypropylcyclodextrin or γ-cyclodextrin), DMSO, ethanol.

The pharmaceutically acceptable carrier, chemotherapeutic agents and compounds of this invention are formulated into unit dosage forms for administration to a patient. The dosage levels of active ingredient (i.e. compounds of this invention) in the unit dosage may be varied so as to obtain an amount of active ingredient effective to achieve lesion-eliminating activity in accordance with the desired method of administration (i.e., oral or rectal). The selected dosage level therefore depends upon the nature of the active compound administered, the route of administration, the desired duration of treatment, individual needs and other factors. If desired, the unit dosage may be such that the daily requirement for active compound is in one dose, or divided among multiple doses for administration, e.g., two to four times per day.

With regard to dosage and duration of treatment according to any aspect of the present invention, it is recognized that the ability of an artisan skilled in pharmaceutical administration of drugs to determine suitable dosages depending on many inter-related factors is well known, and skilled artisans are readily able to monitor patients to determine whether treatment should be started, continued, discontinued or resumed at any given time. For example, dosages of the compounds are suitably determined depending on the individual cases taking symptoms, age and sex of the subject and the like into consideration. The amount of the compound to be incorporated into the pharmaceutical composition of the invention varies with dosage route, solubility of the compound, administration route, administration scheme and the like. An effective amount for a particular patient may vary depending on factors such as the condition being treated, the overall health of the patient and the method, route and dose of administration. The clinician using parameters known in the art makes determination of the appropriate dose. Generally, the dose begins with an amount somewhat less than the optimum dose and it is increased by small increments thereafter until the desired or optimum effect is achieved. Suitable dosages can be determined by further taking into account relevant disclosure in the known art. In one embodiment, the unit dose comprises 5-500 mg of active ingredient consisting of at least one compound of the present invention.

The pharmaceutical formulation of this invention is preferably packaged in a container (e.g. a box or bottle, or both) with suitable printed material (e.g. a package insert) containing indications, directions for use, etc. The pharmaceutical formulations containing the active compounds or pro-drugs of the present invention can be formulated so as to be specifically targeted to tumours and viral, bacterial and/or parasite containing cells. The compounds can be attached to the reagent that is capable of binding a tumour and/or viral, bacterial and/or parasite-associated antigen. For example, the compounds of the present invention could be covalently attached to a monoclonal antibody such as directed to a tumour-associated antigen. The antigen may be located on a tumour/virus/bacterial/parasite or in the tumour/virus cell area. Such linkages can be made through peptide bond formation with amino groups of an antibody. Suitable reagents include polyclonal and monoclonal antibodies. Accordingly, the present invention also provides a method comprising treating cancer and/or viral/bacterial/parasite infections by administering a pharmaceutical formulation of the present invention and a reagent (i.e. monoclonal or polyclonal antibody), which is capable of binding to a tumour/virus/bacterial/parasite associated antigen.

In one embodiment, the liposomes or carbohydrate vehicles are targeted to HIV infected cells by putting viral antibodies on its surface. In another embodiment, the viral antibodies are directed to the HIV coat protein gp160 and/or gp120.

The invention provides a method of treating a neoplasia and/or viral/bacterial/parasite infection and/or suppression of immune response rejection in tissue transplantation comprising administering to a mammal in need thereof an effective amount of a composition containing as an active ingredient therein at least one of the compounds of the present invention, with pharmaceutical acceptable additives, diluents, carriers, excipients and pharmaceutical salts thereof, a chemotherapeutic agent and a pharmaceutically acceptable carrier.

The invention provides a method for manufacturing a medicament for treating neoplasia and/or viral/bacterial/parasite infection and/or suppression of immune response rejection in tissue transplantation wherein the treatment protocol comprises:

Administering a first composition comprising at least one of the compounds of the present invention circiliol, precursor molecules, derivatives, metabolites, analogues and/or mimic molecules with pharmaceutical acceptable additives, diluents, carriers, excipients and pharmaceutical salts thereof and a pharmaceutically acceptable carrier;

later followed by a second composition comprising at least one conventional chemotherapeutic agent and a pharmaceutically acceptable carrier;

during the treatment protocol.

The present invention also provides a method of endowing a chemotherapeutic agent with substantially enhanced therapeutic efficacy and reduced toxicity, comprising:

(a) providing a chemotherapeutic agent; and

(b) combining the agent with one or more compounds of the present invention with pharmaceutical acceptable additives, diluents, carriers, excipients and pharmaceutical salts thereof and a pharmaceutically acceptable carrier thereof so as to reduce the cytotoxicity of the combination in comparison to the chemotherapeutic agent alone.

The invention further provides a pharmaceutical formulation for treating a cancer selected from the group consisting of small cell lung cancer, testicular cancer, lymphoma, leukaemia, oesophageal cancer, stomach cancer, colon cancer, breast cancer, central nervous system cancer, liver cancer and prostate cancer, which comprising administering to a mammal in need thereof an effective amount of a composition containing as an active ingredient therein at least one of the compounds of the present invention, their derivatives, metabolites, analogues and/or mimic molecules with pharmaceutical acceptable additives, diluents, carriers, excipients and pharmaceutical salts thereof.

The invention provides compositions comprising, the compounds of the present invention and at least one chemotherapeutic agent, to be used as anti-viral/bacterial/parasite and/or anti-cancer agents.

The invention provides compositions comprising, the compounds of the present invention and at least one chemotherapeutic agent, to be used in the preparation of medicaments having anti-viral/bacterial/parasite and/or anti-cancer activity.

The invention further provides, the use of the compounds of the present invention and at least one chemotherapeutic agent, is provided as anti-viral/bacterial/parasite and/or anti-cancer agents.

In another embodiment, the use of the compounds of the present invention and at least one chemotherapeutic agent, is provided for the preparation of medicaments having anti-viral/bacterial/parasite and/or anti-cancer activity.

In another embodiment, the compounds of the present invention and at least one chemotherapeutic agent, are provided, for the preparation of medicaments having activity against viral/bacterial/parasite infections and/or cancer.

The invention also relates to a method of suppressing tumour growth in a mammal by administering to the mammal an amount of the compounds of the present invention, and a second chemotherapeutic agent effective to suppress tumour growth in the mammal. The second chemotherapeutic agent is an antimetabolite, nucleotide and/or nucleoside analogue. These compositions provide enhanced antitumour effect and may also prevent the development of metastases. In particular, these compounds are useful for overcoming tumours that are drug resistant. These agents may be administered separately or as a cocktail. Administering the compound of the present invention or a derivative, metabolite, analogue or mimic molecule, thereof several hours prior to administering the chemotherapeutic agent may reduce toxicity. The compositions can be administered by any route.

The components of any of the pharmaceutical formulations disclosed herein can be administered simultaneously (in a combination formulation), essentially simultaneously (e.g., administration of each compound a few minutes or a few hours apart), or can be administered sequentially, e.g., several days apart, or more than a week apart. For example, a compound of the present invention, (and a conventional chemotherapeutic agent) can be administered together, or essentially simultaneously, e.g., administration of each compound a few minutes or a few hours apart, or can be administered sequentially, e.g., several days apart, or more than a week apart. All such variations in administration of the combination therapy are encompassed within the scope of the invention.

Further, the invention provides use of the composition in veterinary medicine, prophylactically and therapeutically in animal populations that are subject to infection that compromises immune response and cause infection.

In another embodiment, the subject or patient is an animal. The term animal as used herein includes, but is not limited to, mice, rats, domesticated animals such as but is not limited to, cats, dogs, and other animals but is not limited to, cattle, sheep, pigs, horses, camels, buffalo and primates such as but not limited to, monkeys, humans and more generally mammals.

Treatment according to the present invention can be effected when the subject or mammal is a neonate. Administration is carried out prior to delivery of the neonate and/or during delivery of the neonate.

Without further elaboration, it is believed that one skilled in the art can, using the preceding description, practice the present invention to its fullest extent. The following detailed examples describe how to test the various compounds of this invention and/or perform the various processes of the invention and are to be construed as merely illustrative, and not limitations of the preceding disclosure in any way whatsoever. Those skilled in the art will promptly recognize appropriate variations from the procedures.

These and other features and advantages of the present invention will be more clearly understood with reference to the following description of some embodiments thereof and with reference to the accompanying drawings in which:

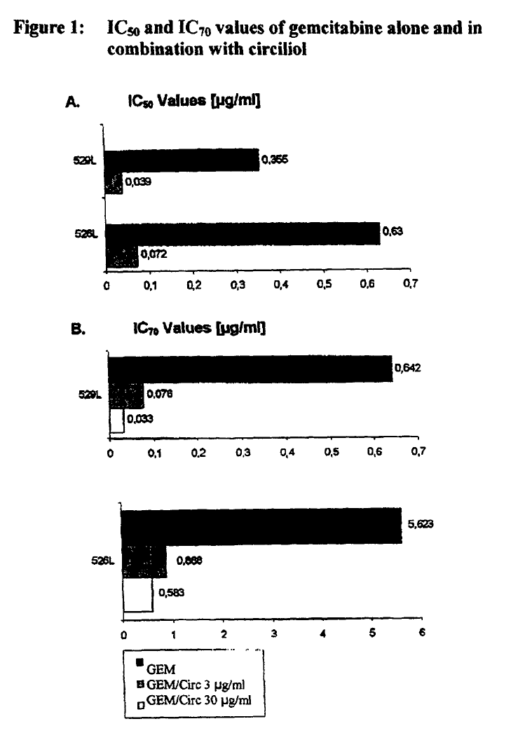

FIG. 1A and B are graphs showing the IC₅₀ and IC₇₀ values of gemcitabine alone and in combination with circiliol respectively; and

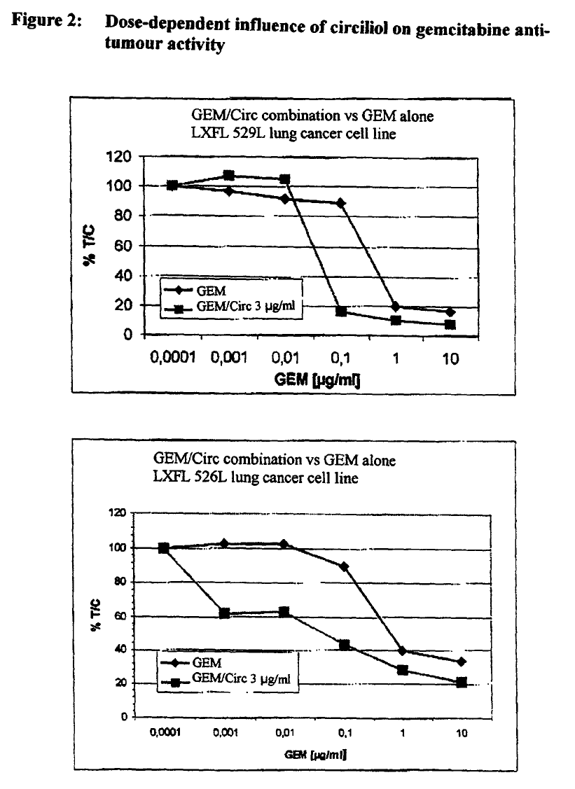

FIG. 2 is graphs showing the dose dependent influence of circiliol on gemcitabine antitumour activity in both cell lines.

### EXAMPLE 1

Gemcitabine (2′, 2′-diflouro-2′-deoxycytidine, dFdC) is approved for the treatment of locally advanced or metastatic pancreatic cancer, with additional activity in non small lung cancer alone and in combination with other cytotoxic agents like 5-flourouracil or cisplatin.

For this study, the in vitro growth of gemcitabine and circiliol, alone and in combination was studied in the large cell lung cancer line LXFL 529L and the lung adenocarcinoma cell line LXFA 526L in a monolayer proliferation inhibition study.

Test Procedure

Cell Lines

The cells were grown in monolayers and routinely passaged weekly. Stocks were not maintained more than 20 passages in vitro. Table 1 summarises the cell line characteristics.

TABLE 1
Characterisation of cell lines

| | | | | Tumour |
|---|---|---|---|---|
| | | Histology in | Doubling | formation |
| Tumour type | Cell line | nude mice | time (hours) | in vivo |
| Lung (NSC) | LXFA 526L | adenocarcinoma | 34 | yes |
| | LXFA 529L | large cell | 25 | yes |

NSC = non small cell

Assay

A modified propidium iodide assay (Dengler et al, 1995) was used to assess the effects of the 2 compounds and the combinations thereof. Cells were harvested from exponential phase cultures growing in RPMI 1640 medium supplemented with 10% foetal calf serum (FCS) and 1% gentamycin (100 U/ml) by tripsination, counted and plated in 96 well flat-bottomed microtitre plates (140 μl cell suspension, 5-20,000 cells/well). After a 24 hour recovery, to allow the cells resume exponential growth, 10 μl of culture medium (6 control wells per plate) or culture medium containing gemcitabine and/or circiliol were added to the wells. Each drug concentration was plated in triplicate. Following 4 days of continuous drug exposure, the culture medium with or without drug was removed and 200 μl of an aqueous propidium iodide solution solution (6 μg/ml) was added which then intercalates into the DNA of the dead cells. Fluorescence was measured using a Cytofluor 2350 microplate reader (excitation 530 nm, emission 620 nm). Growth inhibition was expressed as treated/control×100 (%T/C); IC₅₀ and IC₇₀ values were determined by plotting drug concentration versus cell viability.

Drug Application

Circiliol, a crystalline compound, was dissolved in DMSO at a stock concentration of 100 mg/ml and stored at 4° C. Gemcitabine was used as its clinical formulation (Gemzar, Lilly) and dissolved for each experiment freshly in sterile water at a stock concentration of 3.3 mg/ml.

Results and Discussion

Both cell lines grew very well, with initial cell number increasing 4 fold.

Fluorescence units of controls ranged from 1.421 to 4.277. Clear dose response effects were seen. The determination of antiproliferation effects were repeated up to five times. The in vitro activity of circiliol is summarized in table 2.

TABLE 2
In vitro activity (Test/Control %) of Circiliol

| Concentration of | | | |
|---|---|---|---|
| Circiliol (μg/ml) | 3 | 30 | 300 |
| LXFA 526L | 93 | 53 | 55 |
| LXFA 529L | 102 | 49 | 39 |

Median of 5 Experiments

Circiliol alone was only moderately active in vitro. Concentration of 30 μg/ml and 300 μg/ml resulted in tumour growth inhibition in the range of 39% to 56% versus control in both cell lines. At concentrations of <3 μg/ml circiliol no antitumour activity was evident in vitro.

Gemcitabine was potently active in both cell lines in vitro. The mean IC₅₀ value was 0.63 μg/ml in LXFA 526L adenocarcinoma cell lines and 0.36 μg/ml in the LXFL 529L large cell lung cancer cell lines. The mean IC₇₀ value was 5.7 μg/ml in LXFA 526L and 0.64 μg/ml in LXFL 529L.

Based on these results, gemcitabine was combined with 3 μg/ml circiliol, which showed no tumour effects, and 30 μg/ml circiliol which resulted in 50% inhibition of tumour growth in vitro. The results are presented in tables 3 and 4 and FIGS. 1 and 2.

TABLE 3
IC₅₀ values μg/ml of gemcitabine alone and in combination with
Circiliol

| Concentration of | | | |
|---|---|---|---|
| Circiliol | 0 μg/ml | +3 μg/ml | +30 μg/ml |
| LXFA 526L | 0.63 | 0.072 | n.d. |
| LXFA 529L | 0.355 | 0.039 | n.d. |

IC₅₀ VALUES EVALUATED AS A MEDIAN OF 2-5 EXPERIMENTS PER CELL LINE.
n.d. NOT DETECTABLE

TABLE 4
IC₇₀ values μg/ml of gemcitabine alone and in combination with
Circiliol

| Concentration of | | | |
|---|---|---|---|
| Circiliol | 0 μg/ml | +3 μg/ml | +30 μg/ml |
| LXFA 526L | 5.6 | 0.87 | 0.58 |
| LXFA 529L | 0.64 | 0.076 | 0.033 |

IC₇₀ VALUES EVALUATED AS A MEDIAN OF 2-5 EXPERIMENTS PER CELL LINE.

Treatment of cells with 3 μg/ml circiliol in combination with gemcitabine shifted IC₅₀ values from 0.63 μg/ml to 0.072 μg/ml in LXFA 526L cell lines and from 0.36 μg/ml to 0.04 μg/ml in the LXFA 529L cell lines. IC₇₀ values from 5.6 μg/ml to 0.87 μg/ml in LXFA 526L cell lines and from 0.64 μg/ml to 0.076 μg/ml in the LXFA 529L cell lines. Treatment of cells with 30 μg/ml circiliol in combination with gemcitabine shifted IC₇₀ values from 5.6 μg/ml to 0.58 μg/ml in LXFA 526L cell lines and from 0.64 μg/ml to 0.0033 μg/ml in the LXFA 529L cell lines, corresponding to a 10 and 19 fold increase of antitumour activity respectively.

Circiliol alone demonstrates low activity in no small lung tumour cells LXFA 526L and LXFL 529L. However, when administered in combination with the antimetabolite gemcitabine a 6 to 8 fold increase in antitumour activity of gemcitabine is observed in vitro at a concentration of 3 μg/ml, which is inactive alone.

Pharmaceutically acceptable refers to those properties and/or substances, which are acceptable to the patient from a pharmacological/toxicological point of view including bioavailability and patient acceptance or to the manufacturing chemist from a physical-chemical point of view regarding composition, formulation, stability and isolatability.

The terms “comprise, comprised and comprising” and the terms “include, included and including” are used interchangeably in this specification and are to be afforded the widest interpretation.

The invention is not limited to the embodiments described above, but may be varied in both construction and detail within the scope of the claims.

I claim:

1. A composition comprising:
at least one compound selected from the group of compounds consisting of: circiliol; 6-hydroxy-2,3,4-trimethoxy acetophenone; 2-(3,4-dibenzyloxybenzoyloxy)-4,5,6-trimethoxy acetophenone; 3′4′-dibenzyloxy-2-hydroxy-4,5,6-trimethoxydibenzoyl methane; 6,7-dibenzyloxy-5,6,7-trimethoxy flavone; 3,4-dihydroxy-5,6,7-trimethoxy flavone; 3,4-diacetoxy-5,6,7-trimethoxy flavone, derivatives of said compounds, metabolites of said compounds, analogues of said compounds and/or mimic molecules of said compounds; and
at least one chemotherapeutic agent.

2. A composition as recited in claim 1, wherein said at least one compound is selected from the group consisting of circiliol, precursor molecules of circiliol, derivatives of circiliol, metabolites of circiliol, analogues of circiliol and mimic molecules of circiliol.

3. A composition as recited in claim 1, wherein said at least one chemotherapeutic agent is selected from the group consisting of antiviral compounds, antibacterial compounds, anti-parasitic compounds, anti-cancer compounds and antibiotic compounds.

4. A composition as recited in claim 1, wherein said composition further comprises at least one compound selected from the group consisting of pharmaceutically acceptable additives, diluents, carriers, excipients and pharmaceutically acceptable salts of pharmaceutically acceptable additives, diluents, carriers and excipients.

5. A composition as recited in claim 1, wherein said composition is in the form of a pharmaceutical formulation.

6. A composition as claimed in claim 1, wherein said chemotherapeutic agent is selected from the group consisting of antimetabolites, nucleotide analogues and nucleoside analogues.

7. A composition as claimed in claim 6, wherein said nucleotide analogues are pyrimidine antagonists.

8. A composition as claimed in claim 7, wherein said pyrimidine antagonists are selected from the group consisting of 5-fluorouracil; cytosine arabinoside, and azacitidine.

9. A composition as claimed in claim 6, wherein said nucleotide analogues are purine antagonists.

10. A composition as claimed in claim 9, wherein said purine antagonists are selected from the group consisting of: 6-mercaptopurine; azathioprine; 5-iodo-2′-deoxyuridine; 6-thioguanine; 2-deoxycoformycin, cladribine, cytarabine, fludarabine, mercaptopurine, thioguanine, and pentostatin.

11. A composition as claimed in claim 6, wherein said nucleoside analogues are selected from the group consisting of: purine and pyrimidine nucleosides; ara-nucleosides; amino-nucleosides; and aza-nucleosides.

12. A composition as claimed in claim 6, wherein said nucleoside analogues are selected from the group consisting of AZT (zidovudine); ACV; valacylovir; famiciclovir; acyclovir; cidofovir; penciclovir; ganciclovir; Ribavirin; ddC; ddl (zalcitabine); lamuvidine; Abacavir; Adefovir; Didanosine; gemcitabine; d4T (stavudine); 3TC; BW 1592; PMEA/bis-POM PMEA; ddT, HPMPC, HPMPG, HPMPA, PMEA, PMEG, dOTC; DAPD; Ara-AC, pentostatin; dihydro-5-azacytidine; tiazofurin; sangivamycin; Ara-A (vidarabine); 6-MMPR; 5-FUDR (floxuridine); cytarabine (Ara-C; cytosine arabinoside); 5-azacytidine (azacitidine); HBG [9-(4-hydroxybutyl)guanine], (1S,4R)-4-[2-amino-6-cyclopropyl-amino)-9H-purin-9-yl]-2-cyclopentene-1-methanol succinate (“159U89”), uridine; thymidine; idoxuridine; 3-deazauridine; cyclocytidine; dihydro-5-azacytidine; triciribine, ribavirin, and fludrabine.

13. A composition as claimed in claim 6, wherein said nucleoside analogues are phosphate esters selected from the group consisting of: Acyclovir; 1-β-D-arabinofuranosyl-E-5-(2-bromovinyl)uracil; 2′-fluorocarbocyclic-2′-deoxyguanosine; 6′-fluorocarbocyclic-2′-deoxyguanosine; 1-(β-D-arabinofuranosyl)-5(E)-(2-iodovinyl)uracil; {(1r-1α, 2β, 3α)-2-amino-9-(2,3-bis(hydroxymethyl)cyclobutyl)-6H-purin-6-one}Lobucavir; 9H-purin-2-amine, 9-((2-(1-methylethoxy)-1-((1-methylethoxy)methyl)ethoxy)methyl)-(9Cl); trifluorothymidine; 9->(1,3-dihydroxy-2-propoxy)methylguanine (ganciclovir); 5-ethyl-2′-deoxyuridine; E-5-(2-bromovinyl)-2′-deoxyuridine; 5-(2-chloroethyl)-2′-deoxyuridine; buciclovir; 6-deoxyacyclovir; 9-(4-hydroxy-3-hydroxymethylbut-1-yl)guanine; E-5-(2-iodovinyl)-2′-deoxyuridine; 5-vinyl-1-β-D-arabinofuranosyluracil; 1-β-D-arabinofuranosylthymine; 2′-nor-2′deoxyguanosine; and 1-β-D-arabinofuranosyladenine.

14. A composition as claimed in claim 1, wherein said chemotherapeutic agent is selected from the group of compounds consisting of Chloroquin, primaquine, mefloquine, pyrimethamine-sulfadoxone, atoraquone/dapsone; halofantrine; artemisinin derivatives; atoraquone+proguanol, co-artemether; podophyllotoxin; pentamidine, diloxanide furoate, metronidazole, tindazole, tetracycline, quinacrine, stibogluconate, amphotericin B, quinine, doxycline, trimethoprim-sulfamethoxazole, metronidazole, nifurtimox, suramin, melarsoprol, benznidazole, metabolites of said compounds, salts of said compounds, derivatives of said compounds and any other anti-parasitic agent thereof.

15. A composition as claimed in claim 1, wherein said chemotherapeutic agent is selected from the group consisting of mitomycin C, nalidixic acid, puromycin, sanamycin, and actinomycin.

16. A composition as claimed in claim 1, wherein said chemotherapeutic agent is selected from the group consisting of N⁶-(Δ²-isopentyl) adenosine, N⁶-(Δ²-isopentyl) adenosine-5′-monophosphate, N⁶-(Δ²-isopentyl) adenosine-3′,5′-cyclic monophosphate, benzyladenosine, N⁶-benzyladenosine-5′-monophosphate, N⁶-benzyladenosine-3′,5′-cyclic monophosphate, furfuryladenosine, N⁶-furfuryladenosine-5′-monophosphate, N⁶-furfuryladenosine -3′,5′-cyclic monophosphate, N-(purin-6ylcarbamoyl)-o-chloroaniline ribonucleoside, N-(purin-6ylcarbamoyl)-o-chloroaniline ribonucleoside-5′-monophosphate, N⁶-adamantyladenosine, N⁶-adamantyladenosine-5′-monophosphate, N-(purin-6ylcarbamoyl)-o-octylamine ribonucleoside, N-(purin-6ylcarbamoyl)-o-octylamine ribonucleoside-5′-monophosphate, N-(purin-6ylcarbamoyl)-o-octylamine ribonucleoside-3′,5′-cyclic monophosphate, N⁶-(Δ²-isopentyl)-2-methylthioadenosine, N⁶-(4-hydroxy-3-methyl-trans-2-butenyl)adenosine, N⁶-(3-chloro-trans-2-butenyl) adenosine, surfinal adenosine and preferred metabolites including N⁶-(Δ²-isopentyl) adenine, 6-N-(3-methyl-3-hydroxybutylamino) purine, adenine, hypoxanthine, uric acid, and methylated xanthines.

17. A composition as claimed in claim 1, wherein said at least one chemotherapeutic agent is gemcitabine.

18. A method of treating a patient suffering from neoplasia, comprising administering to said patient:
at least one compound selected from the group of compounds consisting of: circiliol; 6-hydroxy-2,3,4-trimethoxy acetophenone; 2-(3,4-dibenzyloxybenzoyloxy)-4,5,6-trimethoxy acetophenone; 3′4′-dibenzyloxy-2-hydroxy-4,5,6-trimethoxydibenzoyl methane; 6,7-dibenzyloxy-5,6,7-trimethoxy flavone; 3,4-dihydroxy-5,6,7-trimethoxy flavone; 3,4-diacetoxy-5,6,7-trimethoxy flavone, derivatives of said compounds, metabolites of said compounds, analogues of said compounds and/or mimic molecules of said compounds; and
at least one chemotherapeutic agent.

19. A method as recited in claim 18, wherein said at least one compound is selected from the group consisting of circiliol, precursor molecules of circiliol, derivatives of circiliol, metabolites of circiliol, analogues of circiliol and mimic molecules of circiliol.

20. A method as recited in claim 18, wherein said at least one chemotherapeutic agent is selected from the group consisting of antiviral compounds, antibacterial compounds, anti-parasitic compounds, anti-cancer compounds and antibiotic compounds.

21. A method as recited in claim 18, wherein said chemotherapeutic agent is selected from the group consisting of antimetabolites, nucleotide analogues and nucleoside analogues.

22. A method as recited in claim 18, wherein said at least one chemotherapeutic agent is gemcitabine.

23. A method as claimed in claim 18, wherein said at least one compound is contained in a first composition, and said at least one chemotherapeutic agent is contained in a second composition.

24. A method as claimed in claim 23, wherein said first composition is administered to said patient, and later said second composition is administered to said patient.

25. A method as claimed in claim 23, wherein said second composition is administered to said patient, and later said first composition is administered to said patient.

26. A method as claimed in claim 23, wherein said first composition and said second composition are administered to said patient substantially simultaneously.

27. A method as claimed in claim 18, wherein said at least one compound and said at least one chemotherapeutic agent are contained in a single pharmaceutical formulation which is administered to said patient.

28. A method as claimed in claim 18, further comprising administering radiation treatment to said patient.

29. A method as claimed in claim 18, further comprising performing surgery on said patient.

30. A method as claimed in claim 18, wherein said neoplasia is selected from the group consisting of precancerous lesion including syndromes represented by abnormal neoplastic and/or dysplastic, changes of tissue comprising precancerous growths in colonic, breast, renal, central nervous, gastric, or lung tissues, or conditions such as dysplastic nevus syndrome, precursor to malignant melanoma of the skin, dysplastic nevus syndromes, polyposis syndromes, colonic polyps, precancerous lesions of the cervix (i.e., cervical dysplasia), prostatic dysplasia, bronchial dysplasia, breast, bladder and/or skin and related conditions (e.g., actinic keratosis), whether the lesions are clinically identifiable or not.

31. A method as claimed in claim 18, wherein said neoplasia is selected from the group consisting of prostate cancer, colon cancer, small cell lung cancer, large cell lung cancer, lung adenocarcinoma, epidermoid lung cancer, melanoma (including amelanotic subtypes), renal cell carcinoma, gastric carcinoma, cancers of the central nervous system including brain tumours, neuroblastomas, gastric carcinoma, breast cancer, ovarian cancer, testicular cancer, lymphoma and leukaemia, oesophageal cancer, stomach cancer, liver cancers, prostate cancer, cervical cancer, adrenal cancer, oral or mucosal cancer, bladder cancer, pancreatic cancer, lymphoma, Hodgkins disease, sarcomas, Haematopoietic cell cancers such as B cell leukaemia/lymphomas, myelomas, T-cell leukemias/lymphomas, small cell leukemias/lymphomas, null cell, sezary, monocytic, myelomonocytic and Hairy cell leukemias.

32. A method as claimed in claim 18, wherein said neoplasia is in the form of a tumour comprising an epidermoid and myeloid tumour, acute or chronic, nonsmall cell, squamous or solid.

33. A method as claimed in claim 18, wherein said at least one compound is micronized.

34. A method as claimed in claim 18, wherein said at least one compound and/or said chemotherapeutic agent is/are contained in a pharmaceutical formulation which has an enteric coating.

35. A method as claimed in claim 34, wherein each said enteric coating is made of a polymer or copolymer.

36. A method as claimed in claim 35, wherein said polymer or copolymer is selected from the group consisting of poly(lactic-glycolic acid) polyester, cellulose acetate phthalate, hydroxypropyl-methyl cellulose phthalate poly(butyl methacrylate), (2-dimethyl aminoethyl) methacrylate, and methyl methacrylate.

37. A method as claimed in claim 18, wherein said at least one compound and/or said chemotherapeutic agent is/are each administered enterally, parenterally, topically, orally, sub-lingually, rectally, nasally or vaginally.

38. A method as claimed in claim 18, wherein said at least one compound and/or said chemotherapeutic agent is/are each contained in a liposome or a carbohydrate vehicle.

39. A method as claimed in claim 38, wherein said liposome or carbohydrate vehicle is specifically targeted to tumours by covalently attaching a monoclonal antibody directed to a tumour-associated antigen.

40. A method as claimed in claim 18, wherein said at least one compound and/or said chemotherapeutic agent is/are each administered intermittently.

41. A method as claimed in claim 18, wherein said patient is a neonate and said administering is effected prior to delivery of said neonate and/or during delivery of said neonate.

42. A method as claimed in claim 18, wherein said at least one compound acts as a prodrug.

43. A method as recited in claim 18, wherein said at least one compound is administered to said patient in an amount in the range of from 5 to 500 mg.

44. A method as recited in claim 18, wherein said patient is a mammal.

45. A method of treating a patient suffering from viral infection, comprising administering to said patient:
at least one compound selected from the group of compounds consisting of: circiliol; 6-hydroxy-2,3,4-trimethoxy acetophenone; 2-(3,4-dibenzyloxybenzoyloxy)-4,5,6-trimethoxy acetophenone; 3′4′-dibenzyloxy-2-hydroxy-4,5,6-trimethoxydibenzoyl methane; 6,7-dibenzyloxy-5,6,7-trimethoxy flavone; 3,4-dihydroxy-5,6,7-trimethoxy flavone; 3,4-diacetoxy-5,6,7-trimethoxy flavone, derivatives of said compounds, metabolites of said compounds, analogues of said compounds and/or mimic molecules of said compounds; and
at least one chemotherapeutic agent.

46. A method as recited in claim 45, wherein said at least one compound is selected from the group consisting of circiliol, precursor molecules of circiliol, derivatives of circiliol, metabolites of circiliol, analogues of circiliol and mimic molecules of circiliol.

47. A method as recited in claim 45, wherein said at least one chemotherapeutic agent is selected from the group consisting of antiviral compounds, antibacterial compounds, anti-parasitic compounds, anti-cancer compounds and antibiotic compounds.

48. A method as recited in claim 45, wherein said chemotherapeutic agent is selected from the group consisting of antimetabolites, nucleotide analogues and nucleoside analogues.

49. A method as recited in claim 45, wherein said at least one chemotherapeutic agent is gemcitabine.

50. A method as claimed in claim 45, wherein said at least one compound is contained in a first composition, and said at least one chemotherapeutic agent is contained in a second composition.

51. A method as claimed in claim 50, wherein said first composition is administered to said patient, and later said second composition is administered to said patient.

52. A method as claimed in claim 50, wherein said second composition is administered to said patient, and later said first composition is administered to said patient.

53. A method as claimed in claim 50, wherein said first composition and said second composition are administered to said patient substantially simultaneously.

54. A method as claimed in claim 45, wherein said at least one compound and said at least one chemotherapeutic agent are contained in a single pharmaceutical formulation which is administered to said patient.

55. A method as claimed in claim 45, wherein said patient is a mammal.

56. A method as claimed in claim 45, wherein said viral infection is selected from the group consisting of DNA virus infections and RNA virus infections.

57. A method as claimed in claim 56, wherein said DNA virus infections and said RNA virus infections are selected from HIV, SHIV, SIV, FIV, HSV, CMV, HAV, HBV, HCV, HDV, HEV, EBV, BVDV, HSV-1, HSV-2, HSV-6, HHV-6, HHV-8, retrovirus infection, togavirus infection, flavivirus infection, rubivirus infection, pestivirus infection, lipid envelope virus infection, filovirus, picornavirus infection, rhinovirus infection, coronavirus infection, respiratory syncytial virus infection, poliovirus infection, parainfluenza virus infection, influenza virus infection, hantavirus, adeno-associated virus, measles virus, poxvirus, filovirus, human papilloma virus and animal papilloma virus infection.

58. A method as claimed in claim 45, wherein said patient is suffering from one or more complications or co-infections associated with AIDS, AIDS related syndromes, including cachexia and/or wasting syndrome.

59. A method as claimed in claim 45, wherein said at least one compound and/or said chemotherapeutic agent is/are contained in a pharmaceutical formulation which has an enteric coating.

60. A method as claimed in claim 59, wherein said enteric coating is made of a polymer or copolymer.

61. A method as claimed in claim 60, wherein said polymer or copolymer is selected from the group consisting of poly(lactic-glycolic acid) polyester, cellulose acetate phthalate, hydroxypropyl-methyl cellulose phthalate poly(butyl methacrylate), (2-dimethyl aminoethyl) methacrylate, and methyl methacrylate.

62. A method as claimed in claim 45, wherein said at least one compound and/or said chemotherapeutic agent is/are each administered enterally, parenterally, topically, orally, rectally, nasally or vaginally.

63. A method as claimed in claim 45, wherein said at least one compound and/or said chemotherapeutic agent is/are each contained in a liposome or a carbohydrate vehicle.

64. A method as claimed in claim 63, wherein each said liposome or carbohydrate vehicle is targeted to HIV infected cells by putting viral antibodies on a surface of said liposome or carbohydrate vehicle.

65. A method as claimed in claim 64, wherein said viral antibodies are directed to HIV coat protein gp160 and/or gp120.

66. A method as claimed in claim 45, wherein said at least one compound and/or said chemotherapeutic agent is/are each administered intermittently.

67. A method as claimed in claim 45, wherein said patient is a neonate and said administering is effected prior to delivery of said neonate and/or during delivery of said neonate.

68. A method as claimed in claim 45, wherein said at least one compound acts as a prodrug.

69. A method of treating a patient suffering from parasite infection condition, comprising administering to said patient:
at least one compound selected from the group of compounds consisting of: circiliol; 6-hydroxy-2,3,4-trimethoxy acetophenone; 2-(3,4-dibenzyloxybenzoyloxy)-4,5,6-trimethoxy acetophenone; 3′4′-dibenzyloxy-2-hydroxy-4,5,6-trimethoxydibenzoyl methane; 6,7-dibenzyloxy-5,6,7-trimethoxy flavone; 3,4-dihydroxy-5,6,7-trimethoxy flavone; 3,4-diacetoxy-5,6,7-trimethoxy flavone, derivatives of said compounds, metabolites of said compounds, analogues of said compounds and/or mimic molecules of said compounds; and
at least one chemotherapeutic agent.

70. A method as recited in claim 69, wherein said at least one compound is selected from the group consisting of circiliol, precursor molecules of circiliol, derivatives of circiliol, metabolites of circiliol, analogues of circiliol and mimic molecules of circiliol.

71. A method as recited in claim 69, wherein said at least one chemotherapeutic agent is selected from the group consisting of antiviral compounds, antibacterial compounds, anti-parasitic compounds, anti-cancer compounds and antibiotic compounds.

72. A method as recited in claim 69, wherein said chemotherapeutic agent is selected from the group consisting of antimetabolites, nucleotide analogues and nucleoside analogues.

73. A method as recited in claim 69, wherein said at least one chemotherapeutic agent is gemcitabine.

74. A method as claimed in claim 69, wherein said at least one compound is contained in a first composition, and said at least one chemotherapeutic agent is contained in a second composition.

75. A method as claimed in claim 74, wherein said first composition is administered to said patient, and later said second composition is administered to said patient.

76. A method as claimed in claim 74, wherein said second composition is administered to said patient, and later said first composition is administered to said patient.

77. A method as claimed in claim 74, wherein said first composition and said second composition are administered to said patient substantially simultaneously.

78. A method as claimed in claim 69, wherein said at least one compound and said at least one chemotherapeutic agent are contained in a single pharmaceutical formulation which is administered to said patient.

79. A method as claimed in claim 69, wherein said patient is a mammal.

80. A method as claimed in claim 69, wherein said parasite infection is selected from the group consisting of Trypanosoma, Plasmodium, Entamoeba, Balantidium, Leishmania, Pneumocystis, Trichomoniasis, and Toxoplasma.

81. A method as claimed in claim 80, wherein said Trypanosoma, Plasmodium, Entamoeba, Balantidium, Leishmania, Pneumocystis, Trichomoniasis, and Toxoplasma are selected from the group consisting of Trypanosoma cruzi, Trypanosoma brucei, Trypanosoma gambiense, Trypanosoma rhodesiense, Plasmodium falciparum, Plasmodium vivax, Plasmodium malariae, Plasmodium ovale, Plasmodium berghei, Entamoeba histolytica, Balantidium coli, Leishmania brazilienis, Leishmania mexicana, Leishmania donovani, Leishmania tropica, Pneumocystis carinii Trichomoniasis vaginalis, and Toxoplasma gondii.

82. A method as claimed in claim 69, wherein said patient is suffering from a condition selected from the group consisting of malaria, sleeping sickness, African trypanosomiasis, Chagas disease, American trypanosomiasis, cryptosporidiosis, amebiasis, balantidiasis, giardiasis, leishmaniasis, pneumocystosis, trichomoniasis, and toxoplasmosis.

83. A method as claimed in claim 69, wherein said at least one compound and/or said chemotherapeutic agent is/are contained in a pharmaceutical formulation which has an enteric coating.

84. A method as claimed in claim 83, wherein each said enteric coating is made of a polymer or copolymer.

85. A method as claimed in claim 84, wherein said polymer or copolymer is selected from the group consisting of poly(lactic-glycolic acid) polyester, cellulose acetate phthalate, hydroxypropyl-methyl cellulose phthalate poly(butyl methacrylate), (2-dimethyl aminoethyl) methacrylate, and methyl methacrylate.

86. A method as claimed in claim 69, wherein said at least one compound and/or said chemotherapeutic agent is/are each administered enterally, parenterally, topically, orally, rectally, nasally or vaginally.

87. A method as claimed in claim 69, wherein said at least one compound and/or said chemotherapeutic agent is/are each contained in a liposome or a carbohydrate vehicle.

88. A method as claimed in claim 69, wherein said at least one compound and/or said chemotherapeutic agent is/are each administered intermittently.

89. A method as claimed in claim 69, wherein said patient is a neonate and said administering is effected prior to delivery of said neonate and/or during delivery of said neonate.

90. A method as claimed in claim 69, wherein said at least one compound acts as a prodrug.

91. A method of treating a patient suffering from bacterial infection, comprising administering to said patient:
at least one compound selected from the group of compounds consisting of: circiliol; 6-hydroxy-2,3,4-trimethoxy acetophenone; 2-(3,4-dibenzyloxybenzoyloxy)-4,5,6trimethoxy acetophenone; 3′4′-dibenzyloxy-2-hydroxy-4,5,6-trimethoxydibenzoyl methane; 6,7-dibenzyloxy-5,6,7-trimethoxy flavone; 3,4-dihydroxy-5,6,7-trimethoxy flavone; 3,4-diacetoxy-5,6,7-trimethoxy flavone, derivatives of said compounds, metabolites of said compounds, analogues of said compounds and/or mimic molecules of said compounds; and
at least one chemotherapeutic agent.

92. A method as recited in claim 91, wherein said at least one compound is selected from the group consisting of circiliol, precursor molecules of circiliol, derivatives of circiliol, metabolites of circiliol, analogues of circiliol and mimic molecules of circiliol.

93. A method as recited in claim 91, wherein said at least one chemotherapeutic agent is selected from the group consisting of antiviral compounds, antibacterial compounds, anti-parasitic compounds, anti-cancer compounds and antibiotic compounds.

94. A method as recited in claim 91, wherein said chemotherapeutic agent is selected from the group consisting of antimetabolites, nucleotide analogues and nucleoside analogues.

95. A method as recited in claim 91, wherein said at least one chemotherapeutic agent is gemcitabine.

96. A method as claimed in claim 91, wherein said at least one compound is contained in a first composition, and said at least one chemotherapeutic agent is contained in a second composition.

97. A method as claimed in claim 96, wherein said first composition is administered to said patient, and later said second composition is administered to said patient.

98. A method as claimed in claim 96, wherein said second composition is administered to said patient, and later said first composition is administered to said patient.

99. A method as claimed in claim 96, wherein said first composition and said second composition are administered to said patient substantially simultaneously.

100. A method as claimed in claim 91, wherein said at least one compound and said at least one chemotherapeutic agent are contained in a single pharmaceutical formulation which is administered to said patient.

101. A method as claimed in claim 91, wherein said patient is a mammal.

102. A method as claimed in claim 91, wherein said bacterial infection is an intracellular bacterial infection or an extracellular bacterial infection.

103. A method as claimed in claim 91, wherein said bacterial infection is selected from the group consisting of mycoplasma infection, Listeria infection or Mycobacterium infection; Streptococcus infection, Staphylococcus infection, Vibrio infection, Salmonella infection; Shigella infection, enterotoxigenic, enteropathogenic, enteroinvasive or enterohemorrhagic E. coli infection, Yersinia infection, Campylobacter infection, Pseudomonas infection, Borrelia infection, Legionella infection and Haemophilus infection; pulmonary Aspergillosis, mucosal or oropharyngealcandidiasis and juvenile paracoccidiomyosis; and any combinations thereof.

104. A method as claimed in claim 91, wherein said at least one compound and/or said chemotherapeutic agent is/are contained in a pharmaceutical formulation which has an enteric coating.

105. A method as claimed in claim 104, wherein said enteric coating is made of a polymer or copolymer.

106. A method as claimed in claim 105, wherein said polymer or copolymer is selected from the group consisting of poly(lactic-glycolic acid) polyester, cellulose acetate phthalate, hydroxypropyl-methyl cellulose phthalate poly(butyl methacrylate), (2-dimethyl aminoethyl) methacrylate, and methyl methacrylate.

107. A method as claimed in claim 91, wherein said at least one compound and/or said chemotherapeutic agent is/are each administered enterally, parenterally, topically, orally, rectally, nasally or vaginally.

108. A method as claimed in claim 91, wherein said at least one compound and/or said chemotherapeutic agent is/are each contained in a liposome or a carbohydrate vehicle.

109. A method as claimed in claim 91, wherein said at least one compound and/or said chemotherapeutic agent is/are each administered intermittently.

110. A method as claimed in claim 91, wherein said patient is a neonate and said administering is effected prior to delivery of said neonate and/or during delivery of said neonate.

111. A method as claimed in claim 91, wherein said at least one compound acts as a prodrug.

112. A method of treating a patient for suppression of immune response rejection in tissue transplantation, comprising administering to said patient:
at least one compound selected from the group of compounds consisting of: circiliol; 6-hydroxy-2,3,4-trimethoxy acetophenone; 2-(3,4-dibenzyloxybenzoyloxy)-4,5,6-trimethoxy acetophenone; 3′4′-dibenzyloxy-2-hydroxy-4,5,6-trimethoxydibenzoyl methane; 6,7-dibenzyloxy-5,6,7-trimethoxy flavone; 3,4-dihydroxy-5,6,7trimethoxy flavone; 3,4-diacetoxy-5,6,7-trimethoxy flavone, derivatives of said compounds, metabolites of said compounds, analogues of said compounds and/or mimic molecules of said compounds; and
at least one chemotherapeutic agent.

113. A method as recited in claim 112, wherein said at least one compound is selected from the group consisting of circiliol, precursor molecules of circiliol, derivatives of circiliol, metabolites of circiliol, analogues of circiliol and mimic molecules of circiliol.

114. A method as recited in claim 112, wherein said at least one chemotherapeutic agent is selected from the group consisting of antiviral compounds, antibacterial compounds, anti-parasitic compounds, anti-cancer compounds and antibiotic compounds.

115. A method as recited in claim 112, wherein said chemotherapeutic agent is selected from the group consisting of antimetabolites, nucleotide analogues and nucleoside analogues.

116. A method as recited in claim 112, wherein said at least one chemotherapeutic agent is gemcitabine.

117. A method as claimed in claim 112, wherein said at least one compound is contained in a first composition, and said at least one chemotherapeutic agent is contained in a second composition.

118. A method as claimed in claim 117, wherein said first composition is administered to said patient, and later said second composition is administered to said patient.

119. A method as claimed in claim 117, wherein said second composition is administered to said patient, and later said first composition is administered to said patient.

120. A method as claimed in claim 117, wherein said first composition and said second composition are administered to said patient substantially simultaneously.

121. A method as claimed in claim 112, wherein said at least one compound and said at least one chemotherapeutic agent are contained in a single pharmaceutical formulation which is administered to said patient.

122. A method as claimed in claim 112, wherein said patient is a mammal.

123. A method as claimed in claim 112, wherein said at least one compound and/or said chemotherapeutic agent is/are contained in a pharmaceutical formulation which has an enteric coating.

124. A method as claimed in claim 123, wherein said enteric coating is made of a polymer or copolymer.

125. A method as claimed in claim 124, wherein said polymer or copolymer is selected from the group consisting of poly(lactic-glycolic acid) polyester, cellulose acetate phthalate, hydroxypropyl-methyl cellulose phthalate poly(butyl methacrylate), (2-dimethyl aminoethyl) methacrylate, and methyl methacrylate.

126. A method as claimed in claim 112, wherein said at least one compound and/or said chemotherapeutic agent is/are administered enterally, parenterally, topically, orally, rectally, nasally or vaginally.

127. A method as claimed in claim 112, wherein at least one compound and/or said chemotherapeutic agent is/are contained in a liposome or a carbohydrate vehicle.

128. A method as claimed in claim 112, wherein said at least one compound and/or said chemotherapeutic agent is/are administered intermittently.

129. A method as claimed in claim 112, wherein said patient is a neonate and said administering is effected prior to delivery of said neonate and/or during delivery of said neonate.

130. A method as claimed in claim 112, wherein said at least one compound acts as a prodrug.

131. A method of endowing a chemotherapeutic agent with substantially enhanced therapeutic efficacy and reduced toxicity, comprising combining a chemotherapeutic agent with at least one compound selected from the group of compounds consisting of: circiliol; 6-hydroxy-2,3,4-trimethoxy acetophenone; 2-(3,4-dibenzyloxybenzoyloxy)-4,5,6-trimethoxy acetophenone; 3′4′-dibenzyloxy-2-hydroxy-4,5,6-trimethoxydibenzoyl methane; 6,7-dibenzyloxy-5,6,7-trimethoxy flavone; 3,4-dihydroxy-5,6,7-trimethoxy flavone; 3,4-diacetoxy-5,6,7-trimethoxy flavone, derivatives of said compounds, metabolites of said compounds, analogues of said compounds and/or mimic molecules of said compounds,
thereby reducing the cytotoxicity of said chemotherapeutic agent in comparison to said chemotherapeutic agent alone.

132. A method as recited in claim 131, wherein said chemotherapeutic agent is gemcitabine.

133. A method as recited in claim 131, wherein said at least one compound is selected from the group consisting of circiliol, precursor molecules of circiliol, derivatives of circiliol, metabolites of circiliol, analogues of circiliol and mimic molecules of circiliol.

134. A method as recited in claim 132, wherein said at least one compound is selected from the group consisting of circiliol, precursor molecules of circiliol, derivatives of circiliol, metabolites of circiliol, analogues of circiliol and mimic molecules of circiliol.

135. A kit comprising:
unit dosages of at least one compound selected from the group of compounds consisting of: circiliol; 6-hydroxy-2,3,4-trimethoxy acetophenone; 2-(3,4-dibenzyloxybenzoyloxy)-4,5,6-trimethoxy acetophenone; 3′4′-dibenzyloxy-2-hydroxy-4,5,6-trimethoxydibenzoyl methane; 6,7-dibenzyloxy-5,6,7-trimethoxy flavone; 3,4-dihydroxy-5,6,7-trimethoxy flavone; 3,4-diacetoxy-5,6,7-trimethoxy flavone, derivatives of said compounds, metabolites of said compounds, analogues of said compounds and/or mimic molecules of said compounds, and
unit dosages of at least one chemotherapeutic agent.

136. A kit as recited in claim 135, wherein said at least one compound is selected from the group consisting of circiliol, precursor molecules of circiliol, derivatives of circiliol, metabolites of circiliol, analogues of circiliol and mimic molecules of circiliol.

137. A kit as recited in claim 135, wherein said at least one chemotherapeutic agent is selected from the group consisting of antiviral compounds, antibacterial compounds, anti-parasitic compounds, anti-cancer compounds and antibiotic compounds.

138. A kit as recited in claim 135, wherein said chemotherapeutic agent is selected from the group consisting of antimetabolites, nucleotide analogues and nucleoside analogues.

139. A kit as recited in claim 135, wherein said at least one chemotherapeutic agent is gemcitabine.
